(19) United States
(12) Patent Application Publication
(10) Pub. No.: US 2002/0120208 A1
(43) Pub. Date: Aug. 29, 2002

(54) System and method of providing service for enhancing attention on internet

(76) Inventors: Sun-Il Kim, Seoul (KR); Jang Han Lee, Anyang-si (KR); Dong Joo Kang, Seoul (KR); Dong-Pyo Jang, Sungnam-city (KR); In-Young Kim, Seoul (KR); Jae-Seok Kim, Seoul (KR); Jeong-Hun Ku, Pusan (KR); Back-Hwan Cho, Pusan (KR)

Correspondence Address: LOWE HAUPTMAN GOPSTEIN, GILMAN AND BERNER LLP, SUITE 310, 1700 DIAGONAL ROAD, ALEXANDRIA, VA, 22314

(21) Appl. No.: 10/078,421

(22) Filed: Feb. 21, 2002

(30) Foreign Application Priority Data
Feb. 23, 2001 (KR) 2001-0009134

(51) Int. Cl. A61B 5/0482

(52) U.S. Cl. 600/545

(57) ABSTRACT

The present invention discloses a method and system of network psychiatric diagnosis and therapy for patients suffering from attention deficit disorder.

The present invention comprises a biofeedback sensor for evaluating the status and progress of the trainee's exercise.

### FIELD OF THE INVENTION

The present invention relates to attention deficit disorder (ADD) or psychiatric treatment for enhancing the attention of a patient on network. The present invention discloses an on-line biofeedback technique for enhancing the attention level of the patient and more particularly to a system and method for diagnosing, monitoring, presenting psychiatric exercise contents on Internet, and thereby enhancing the attention level.

### DESCRIPTION OF THE RELATED ART

Recent rapid change in the pattern of modern life has resulted in a variety of psychoses. The deficiency of attention can be thought of as a critical loss not only to the personal point of view but also to the society.

Attention of a human being is a fundamental recognizing capability and the attention deficit disorder (ADD) results in difficulty in mental absorption, listening, and memorizing activities.

In other words, the psychiatric patients suffering from the attention deficit disorder (ADD) tend to become inattentive, impulsive, emotionally unstable, and sometimes excessively overactive.

The above-mentioned symptoms give rise to problems in studying, working, completing the assigned task, and keeping good company with friends. The attention enhancement system is a system for therapy or treatment of patients suffering from ADD.

A conventional therapy for ADD can be a drug-based treatment such as prescribing some drug like retalin. Another approach for the treatment of ADD is a bio-graph approach employing a biofeedback.

The above-mentioned conventional treatments, however, have shortcomings in that the patient should visit a clinic (hospital) in order to have a doctor's diagnosis and subsequent treatment.

Therefore, it will be very much convenient if the diagnosis and/or the therapy can be provided to the patient on network like Internet.

### SUMMARY OF THE INVENTION

Therefore, there is a need in the art for a psychiatric therapy system and method for the treatment the attention deficit disorder and/or just for enhancing the attention level of the trainee on network like Internet.

Accordingly, it is an object of the present invention to provide an efficient therapy and diagnosis system and method for enhancing attention of a trainee on the network.

Yet it is another object of the present invention to provide a therapy and diagnosis system and method for enhancing attention of a human being both on network and in reality (offline).

Another object of the present invention is to provide a diagnosis and therapy system and method having a biofeedback device to monitor the attention level of the trainee in real time on the network.

In accordance with a broad aspect of the present invention, there is provided a network therapy system and method of presenting a medical service of attention enhancement on the network including Internet.

### BRIEF DESCRIPTION OF THE DRAWINGS

FIG. 1 is a schematic diagram illustrating a preferred embodiment of a network therapy system for enhancing attention of a patient (or a trainee) in accordance with the present invention.

FIG. 2 is a schematic diagram illustrating an embodiment of a network therapy method for enhancing attention of a patient (or a trainee) in accordance with the present invention.

FIG. 3 is a schematic diagram illustrating an embodiment of both on-line and off-line therapy for enhancing attention of a patient (or a trainee).

### DETAILED DESCRIPTION OF THE PREFERRED EMBODIMENT OF THE INVENTION

Korean Patent Application No. 10-2001 -009134 filed on Feb. 23, 2001 is incorporated herein by reference as it fully set forth herein. The present invention will be explained in detail with reference to the accompanying drawings.

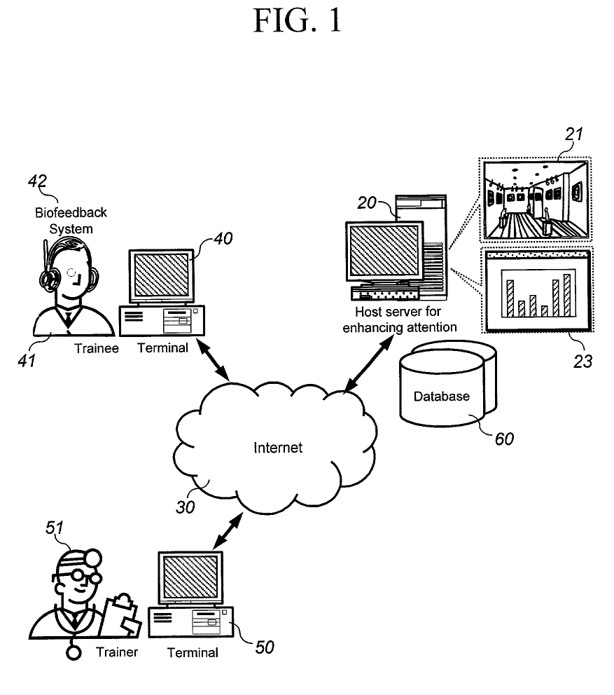

FIG. 1 is a schematic diagram illustrating a preferred embodiment of a network therapy system for enhancing the attention level and/or for curing the attention deficit disorder (ADD) in accordance with the present invention.

Referring to FIG. 1, a patient (or a trainee) 41 performs a mental exercise through appreciating and enjoying the contents 21 that are provided from a homepage of a host server 20 on the web. In the meanwhile, the measured real-time data regarding the patient's mental status is sent to a host server 20 and stored at a database 60.

A trainer (or a doctor, an expert, whatever it may be called) 51 can be consulted by the patient 41 on the web. In other words, the doctor 51 refers to the database 60 for the analysis of the patient's mental status and delivers some advice to the patient 41 on the web.

As a preferred embodiment, the patient 41 can refer to a scheduled program for therapy as well as the plot, for instance, depicting the progress of his psychiatric exercise in real-time by looking at his monitor 40 hooked on the network.

Preferably, the network attention-enhancement therapy system in accordance with the present invention can be implemented with cable television (CATV) network, satellite communication network, wireless network, and PSTN network as well as the Internet.

Referring to FIG. 1 again, the patient (or the trainee) 41 can make an appointment with the doctor 51 on Internet 30, and then have a more elaborate face-to-face diagnosis and intensive therapy with the doctor 51 in reality.

Additionally, the patient can take a prescription for mental therapy from the web page provided by a host server 20 on Internet.

In the meanwhile, the contents for enhancing attention including the treatment of ADD of the trainee is provided in real time by the application software installed at the host server 20.

Consequently, the patient or the trainee 41 can do mental/psychiatric exercise without installing the application software at his own computer but simply by visiting the web page of the host computer 20.

In addition, the patient or the trainee 41 can enjoy a variety of psychiatric services from the host server 20 on Internet.

As a preferred embodiment in accordance with the present invention, the information data about the trainee and the trainee's progress in therapy is administrated at a database server 60 and the trainee 41 can observe the progress report on his monitor.

Preferably, the doctor or the trainer 51 checks the improvement of the level of the patient's attention simply by referring to the database 60 of the host server 20.

As a preferred embodiment in accordance with the present invention, the trainee or the patient 41 can consult the doctor or the trainer 51 in reality.

The diagnosis after the consultation is stored at database of each patient's home page and then the therapy for enhancing attention is provided on the network.

The trainer 51 evaluates the progress of the patient's mental exercise on the network. The evaluation is reported to the trainee 41. The exercise thereby can be more effectively performed through a regular meeting between the trainer and the trainee in reality.

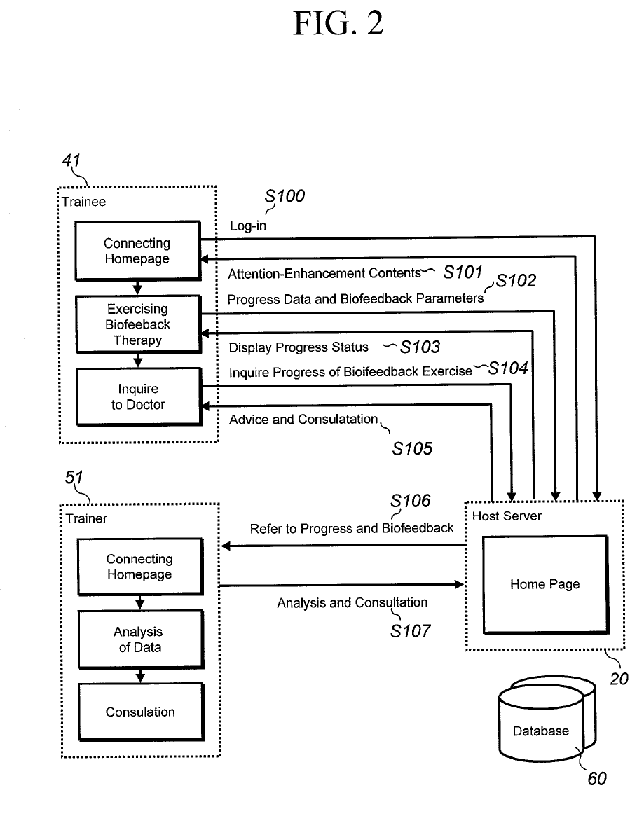

FIG. 2 is schematic diagram illustrating a service scenario for network therapy in accordance with the present invention.

Referring to FIG. 2, the patient or the trainee 41 can be connected to a host server 20 providing psychiatric exercise contents for enhancing attention at his local computer terminal 40(step S100).

Then the contents for on-line psychiatric therapy are provided to the trainee 41 (step S101).

As a preferred embodiment in accordance with the present invention, a three-dimensional virtual reality or a two-dimensional animation can be employed.

More preferably, the computer environment for network therapy can include a two-dimensional animation.

The three-dimensional virtual reality (VR) in accordance with the present invention can comprise a three-dimensional web VR that enables the computer user to experience the three-dimensional world on the web.

Preferably, a three-dimensional software development tool such as JAVA 3D or VRML can be employed to implement VR.

As an exemplary VR for psychiatric therapy for enhancing attention of the ADD patient, a classroom can be chosen.

In this VR for the classroom, a large screen for displaying various kinds of motion pictures is prepared as well as avatar representing a teacher.

In front of the trainee, there is provided a desk on which the trainee can do the exercises for the enhancement of his attention.

As another preferred embodiment in accordance with the present invention, the two-dimensional graphic environment for implementing the network psychiatric therapy system can include animation.

As a consequence, the exercise contents also allow the trainee to communicate with the host computer 20.

In other words, the status record during the trainee's exercise is sent to a host computer as well as the biofeedback parameters. As a consequence, it makes it possible that the trainee does a mental exercise on network as if he is in a real training clinic.

As a preferred embodiment in accordance to the present invention, the trainee 41 can use other equipment including an HMD (head-mounted display), tracker, a three-dimensional speaker, and a data glove.

More preferably, a biofeedback sensor can be employed to evaluate the level of the trainee's attention during the psychiatric exercise on network.

As a preferred embodiment of the biofeedback sensor in accordance with the present invention, the electroencephalogram of the trainee can be recorded and sent to the host server 20 on network.

The biofeedback sensor can evaluate the level of the trainee's attention by extracting the SMR-wave (12^{˜}16 Hz), beta wave (16^{˜}20 Hz), and theta wave (4^{˜}8 Hz), and calculating the relative ratio of strength for each brain wave.

As a preferred embodiment in accordance with the present invention, the attention-enhancement contents for the treatment of ADD can be implemented with a software tool including FLASH or JAVA.

Preferably, the attention-enhancement contents can be prepared with subjects for improving the trainee's attention, sustained attention, selective attention, and attention shift.

As a preferred embodiment in accordance with the present invention, the subjects of the attention-enhancement contents can be a visual comparison module, an auditory comparison module, a memory comparison module, and attention shifting module.

The biofeedback technique in accordance with the present invention relies on a scheme of extracting bio-parameters of brain waves that are related to attention, followed by the normalization of the patient's attention level.

Advising the level of the attention and consequently the progress of the exercise on the web, the doctor or the trainee can make a plan for next-step therapy for the patient.

Simultaneously, the patient or the trainee can observe the status record of the psychiatric exercise on the web through the biofeedback.

As a preferred embodiment, a biofeedback sensor 42 is prepared at a trainee's terminal 40 and the trainee performs the bio-feed exercise in accordance with the displayed contents at his terminal 40 (steps S102 and S103).

The biofeedback data is transmitted to the host computer 20 in real-time and the host computer 20 analyzes the biofeedback. Thereafter, the host computer 20 updates the exercise contents in accordance to the result of the analysis of the biofeedback data.

In the meanwhile, the trainee can observe the progress level of his attention therapy on his monitor.

Preferably, the trainer or the doctor 51 can have an access to the host server 20 and fetch the analysis data of the trainee for giving some advice (step S107).

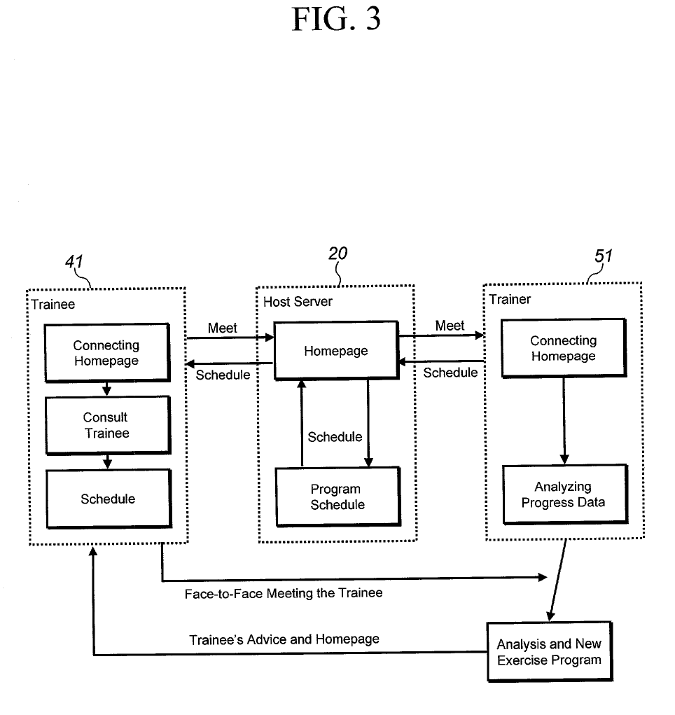

FIG. 3 is a schematic diagram illustrating an embodiment of an on/off-line therapy method for enhancing attention of the patient.

Referring to FIG. 3, the trainee 41 can have an access to a host server 20 and make an inquiry if he can see and consult the doctor in reality. The trainer or the doctor can prepare for the consultation by referring to the schedule of the psychiatric exercise of the patient.

Although the invention has been illustrated and described with respect to exemplary embodiments thereof, it should be understood by those skilled in the art that various other changes, omissions and additions may be made therein and thereto, without departing from the spirit and scope of the present invention.

Therefore, the present invention should not be understood as limited to the specific embodiment set forth above but to include all possible embodiments which can be embodies within a scope encompassed and equivalents thereof with respect to the feature set forth in the appended claims.

What is claimed is:

1. A method of providing a network psychiatric therapy to a trainee for enhancing the attention of the trainee sitting in front of a computer terminal, comprising steps of:
(a) presenting psychiatric exercise contents for enhancing the trainee's attention at the trainee's terminal visiting the home page of a host server;
(b) sending a bio-feedback data including the trainee's brain waves from the trainee's terminal to the host server while the trainee performs the psychiatric exercise by enjoying the contents on the web; and;
(c) updating psychiatric exercise contents for the trainee in accordance with the analysis on the current bio-feedback data of the trainee as well as the progress data of the trainee's exercise and thereby providing the analysis data to the trainee on the web.

2. The method as set forth in claim 1 wherein said network comprises of either one or the combinations from the group of Internet, cable television network, satellite communication network, wireless communication network, and PSTN.

3. The method as set forth in claim 1 wherein said step (b) further comprises steps of:
(a) storing said biofeedback data and the trainee's progress report at a database; and
(b) having a trainer get an access to the data about the trainee for the analysis and advice.

4. The method as set forth in claim 1 wherein said contents comprise a three-dimensional virtual reality or a two-dimensional animation.

5. The method as set forth in claim 1 wherein said biofeedback data of the trainee's brain wave comprises either one or the combination from the group of an SMR-wave, a beta wave, and a theta wave.

6. The method as set forth in claim 4 wherein said two-dimensional animation comprises either one or the combination from the group of attention, sustained attention, selective attention, attention shift, and scattered attention, while the subject comprises either one or the combination from the group of a visual comparison module, auditory comparison module, memory comparison module, and attention shifting module.

7. A network therapy system for psychiatric treatment comprising:
(a) a host server providing psychiatric exercise contents for enhancing attention to a trainee's terminal visiting the homepage and receiving a bio-feedback data and a progress report of the trainee's psychiatric exercise from the terminal;
(b) a trainee's computer terminal displaying the contents to said trainee and sending the bio-feedback data including the trainee's brain wave data to said host server;
(c) a database server storing the progress record of the trainee's exercise and the bio-feedback data;
(d) a trainer's computer terminal wherein said trainer provides the prescription or the advise either on-line or off-line upon the analysis of the data of the trainee; and
(e) a network that connects said trainee's computer terminal, said host server, said database server, and said trainer's computer terminal.

8. The system as set forth in claim 7 wherein said network comprises of either one or the combinations from the group of Internet, cable television network, satellite communication network, wireless communication network, and PSTN.
